(19) United States
(12) Patent Application Publication
(10) Pub. No.: US 2017/0188913 A1
(43) Pub. Date: Jul. 6, 2017

(54) Method for Extracting Fluids from Tissue and Uses Thereof

(71) Applicant: Path Scientific, LLC, Carlisle, MA (US)

(72) Inventors: Terry O. Herndon, Carlisle, MA (US); Thiruvallur R. Gowrishankar, Acton, MA (US)

(21) Appl. No.: 14/983,759

(22) Filed: Dec. 30, 2015

(51) Int. Cl. A61B 5/145; A61B 5/15

(52) U.S. Cl. CPC A61B 5/14532; A61B 5/150198; A61B 5/150358; A61B 5/150458

(57) ABSTRACT

The present invention relates to methods and devices for extraction of bodily fluids from tissue by creating microscopic openings in the outermost layers of the skin and drawing out the fluid. One embodiment of the invention is a cylindrical hollow member, made of electrically conducting material, with sharpened edges.

### FIELD OF THE INVENTION

The present invention relates to a device and methods of using the same for extracting small volumes of fluids from tissues by creating microscopic openings in the outermost layers of the skin and subsequently drawing out the fluid.

### BACKGROUND OF THE INVENTION

Recent developments in technology have enabled simultaneous testing of a variety of health conditions from a single or a few drops of blood. In addition, new areas of testing are envisioned that would facilitate the testing of patient suitability for a new drug or identify gene sequences that indicate the presence of cancer or infection.

Blood glucose measurement is the most widely used test that requires a drop of blood. Such measurements still rely on the piercing of skin by a lancet, squeezing the skin to produce a blood drop, and aligning a glucose test strip to the blood drop. Several such painful procedures are required per day for some patients. Repeated sampling over a long time causes calluses and scarring. In addition, alignment of the small capillary in a glucose strip to the blood drop is challenging for patients with failing vision or compromised mobility.

Current blood glucose measurement techniques involve drawing a drop of blood out by puncturing the skin, and collecting the blood into a chamber inside a glucose strip. The blood then reacts with an enzyme called glucose oxidase which produces an electrical or optical change that is sensed and displayed as a blood glucose level by the electronics inside the glucose meter into which the glucose strip is plugged.

U.S. Pat. No. 8,241,229 describes a drill device known as the PathFormer, and a method of removing the stratum corneum from the epidermis to permit blood collection from the body for analysis, which comprises the steps of drilling into a target area with a drilling assembly, monitoring an electrical impedance of the target area, stopping the drilling into the target area when a change in the electrical impedance is detected, thereby forming a microconduit, and removing blood from the body through the microconduit; wherein the drilling assembly includes a motor, an impedance sensor, and a cutter, and wherein the drilling into the tissue is stopped and automatically reversed by a control module when a change in the electrical impedance of the target area is detected by the sensor. This patent is hereby incorporated herein by reference in its entirety.

Human skin serves to protect the inner body from the outer world. The outermost protective shield of the skin is called the stratum corneum (SC). SC consists of 15 to 20 dead skin cells stacked atop one another, not dissimilar to the stacking of bricks used to form a wall. The thickness of the SC is approximately 4 thousandths of an inch, roughly the thickness of a sheet of paper. The SC is continuously regenerating, shredding its outermost cells and adding newly dead cells at its base from the underlying living cells protecting themselves and all internal tissues within the body.

The SC is highly resistant to the flow of current, in contrast to the much lower resistance and greater conductivity of the live tissue and fluids lying immediately below the SC. By rough analogy, the SC can be considered the rubber insulation surrounding an electrical cord, with that which lies beneath the SC considered as the conductive copper wire, thus protected. The SC has much higher electrical impedance (approximately 2 million ohms) than the underlying tissue (around 20,000 ohms).

The PathFormer technology is predicated on the differential in resistance between the SC and the protected tissues and fluids beneath. This allows the PathFormer device to penetrate and remove the very thin layer of SC without coming into contact with the nerve bed of the underlying live tissue. The device can be programmed to cut, drill or abrade the SC, while continuously measuring impedance. Once the SC is breached, the dramatic change in measured impedance causes the cutting member to retract automatically and immediately. Accordingly, the patient experiences no pain and there is no damage to the living tissues beneath the SC.

Removal of the SC allows for safe, quick and painless delivery of several therapeutic and cosmetic agents and treatments including topical drug delivery of large molecule drugs; rapid delivery of topical anesthetic agents in neonates; treatment of psoriasis; enhanced biometric readings; hair implantation; precision bone drilling and other applications.

The human nail plate is substantially equivalent to the SC and is likewise electrically resistant. Accordingly, the PathFormer device may be programmed to drill microconduits through the nail without the patient experiencing any pain. These microconduits allow true topical delivery of antifungal to treat onychomycosis (nail fungus) and nail psoriasis. A limited clinical trial in a major Boston hospital has proven the efficacy of such treatment. The device can also be used to relieve subungual hematoma (a black toe); an injury commonly suffered by athletes, soldiers, and construction workers. The PathFormer device has obtained FDA 510(k) clearance for such use.

U.S. Patent Pub. No. 2009-0221893 describes a blood glucose measuring device, equipped with a drill device, attachment assembly, and disposable sensing and measurement assembly. The attachment assembly contains an attachment ring that connects to the drill device and is used to hold the disposable sensing and measurement assembly. A detach actuating cam and output shaft are attached to the drill device. Spring tongs are attached to the output shaft by a compression ring further clamp to an end cap. A skin penetrator is attached to the end cap. The disposable sensing and measurement assembly is enclosed in a disposable case. An outer telescoping anti-bend tube is attached to the end cap. An inner anti-bend capillary sensor tube contains analyte sensors and is attached to the disposable case. The electrical conductors for the analyte sensor electrodes are attached to the capillary sensor tube and thus to the disposable case. An impedance sensing electrode on the bottom of the case provides electrical contacts to the skin. The electrical conductor to the impedance sensing electrode is attached to the bottom of the disposable case. This publication is hereby incorporated herein by reference in its entirety.

It is an object of the present invention to provide a device and method that enables blood sampling and analyte measurement in a single procedure. It is an object of the present invention to provide a device and method that reduces or wholly overcomes some or all of the difficulties inherent in prior known devices and methods. Particular objects and advantages of the invention will be apparent to those skilled in the art, that is, those who are knowledgeable or experienced in this field of technology, in view of the following disclosure of the invention and detailed description of certain preferred embodiments.

### SUMMARY OF THE INVENTION

The present invention relates to methods and devices for extraction of bodily fluids from tissue by creating microscopic openings in the outermost layers of the skin (stratum corneum and epidermis) and drawing out internal bodily fluids such as blood and interstitial fluid.

One embodiment of the invention is a sampling and measurement unit comprising an electrically conducting element (cutter) that breaches the stratum corneum and/or the epidermis of the skin, a drive assembly that moves the cutting element repeatedly into and away from the skin, an analyte sensing element, and an electronic control module for controlling the skin cutting element and determining the quantities of one or more analytes in blood.

Another embodiment is a method of extracting bodily fluids, such as and not limited to, blood and interstitial fluid from tissue, which comprises of repeating the following steps: the rotating cutter element is driven toward the skin in a ‘dither’ mode wherein the cutter is moved in small steps toward and away from the skin with a small bias in movement toward the skin, monitoring an electrical impedance, and reversing the direction of movement when a preset level of decrease in the electrical impedance is detected; the procedure is repeated until a predetermined quantity of bodily fluid is collected. Each dither includes both an upward and a downward movement of the cutter. These movements can be adjusted for timing and distance as desired.

As described above, one aspect of the invention is to move the cutter toward the skin in a ‘dither’ mode. When a bodily fluid emerges from the skin, the cutter draws it out using the surface tension between the fluid and the cutter. As the cutter moves toward the skin, it encounters the fluid, electrical impedance drops, and the cutter retracts (moves away from the skin). This causes the cutter to pull the fluid bolus up, thus enlarging the bolus.

One embodiment of the invention is to position the analyte sensing element close to the cutter such that when the fluid is drawn out of the skin, it enters a capillary in the sensing element. This enables the unification of sampling and measurement aspects into a single device.

Another aspect of the invention is to roughen the surface of the cutter to enhance the surface tension effect and improve the extraction of the bodily fluid.

One embodiment of the invention is to have the cutter spinning as it moves towards and away from the skin. The rotating cutter will create an opening in skin large enough to produce a drop of blood or interstitial fluid, and subsequently, draw the blood or interstitial drop out by surface tension.

Another aspect of the invention is to have the cutter spinning until an opening is created in the skin, and subsequently, move toward and away from the skin (dither) to draw the blood or interstitial fluid out by surface tension.

Another aspect of the invention is to have the cutter spinning until an opening is created in the skin, and subsequently, stop the cutter from rotating and move it toward and away from the skin (dither) to draw the blood or interstitial fluid out by surface tension.

Another aspect of the invention is to have a hollow rotating cutter that will dither towards the skin to create an opening and subsequently dither to collect the fluid into the bore of the cutter wherein the fluid contacts the sensing material.

It should be appreciated by those persons having ordinary skill in the art(s) to which the present invention relates that any of the features described herein in respect of any particular aspect and/or embodiment of the present invention can be combined with one or more of any of the other features of any other aspects and/or embodiments of the present invention described herein, with modifications as appropriate to ensure compatibility of the combinations. Such combinations are considered to be part of the present invention contemplated by this disclosure.

It is to be understood that both the foregoing general description and the following detailed description are exemplary and explanatory only and are not restrictive of the invention as claimed. Other embodiments will be apparent to those skilled in the art from consideration of the specification and practice of the invention disclosed herein.

### BRIEF DESCRIPTION OF THE DRAWINGS

FIG. 1 is a schematic of a preferred embodiment of the invention: a cutter 2 in proximity to skin 1. The cutter is driven toward/away from the skin by an up/down drive mechanism. The cutter may be rotated throughout all or part of the process by the rotational drive mechanism. The impedance sensor continually measures the electrical impedance between the cutter 2 and the counterelectrode 3. The electronic control module controls the rotational and translational movement of the cutter. Once a fluid sample is collected, the analyte measurement module determines the concentration of one or more analytes in the fluid. The concentrations of the analytes are displayed by the display module.

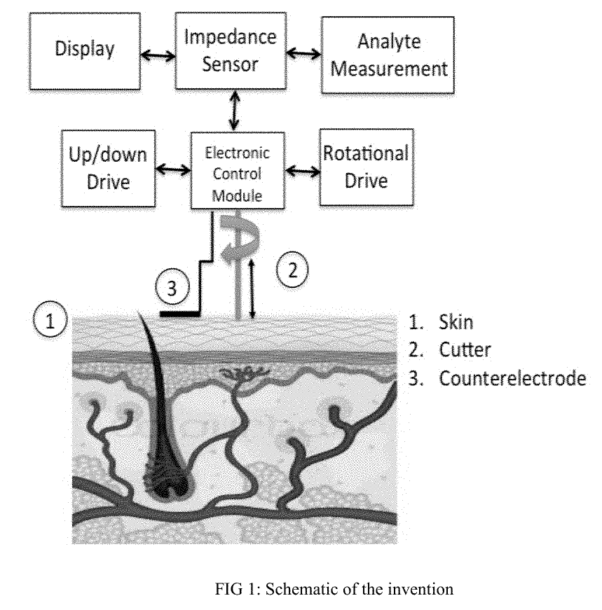

FIG. 2 is a schematic of a preferred embodiment of the blood sampling process. The cutter is driven toward the skin in dither mode (Panel 1). When the skin breaches the stratum corneum of the skin, it encounters the low electrical impedance of the epidermal tissue (Panel 2); it retracts from the skin leaving an opening in the stratum corneum (Panel 3). As the cutter pulls out, interstitial fluid appears out of the opening (Panel 4). As the cutter drives down toward the skin, interstitial fluid/blood flows out of the skin, when the fluid contacts the cutter; low impedance is sensed causing the cutter to retract (Panel 5). During the retraction, surface tension forces enable the cutter to drag the bolus out, enlarging it (Panel 6).

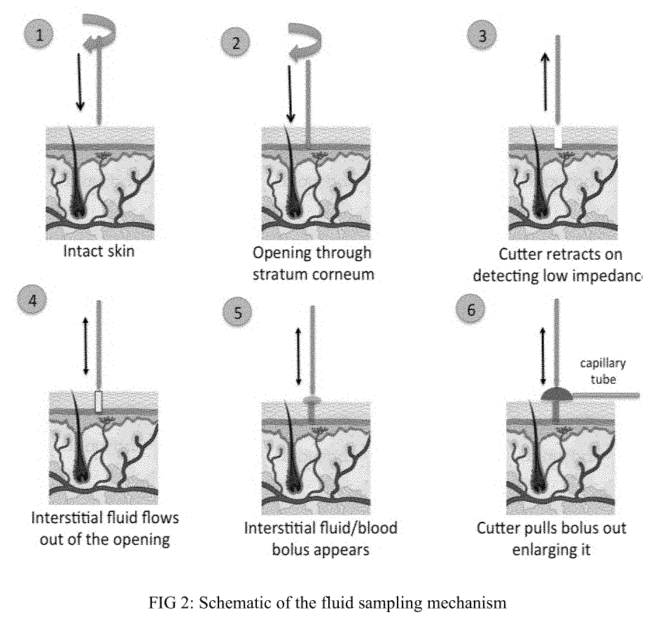

FIG. 3 shows the setup to demonstrate the proof-of-concept of the invention. The device 1 is placed on the back of hand (as an example site). The device 1 has two motors, 3 to drive the cutter 12 toward and away from the skin 18 and 4 to rotate the cutter around its long axis. A lead screw mechanism 8 is used to drive the movable carriage 6. The foot of the device 13 has a foot ring 14 with a central opening that enables the skin to bulge in toward the cutter to create a firm surface for the cutter to contact. An electrically conducting ring 15 is used as a counterelectrode. In certain embodiments a pair of ECG electrodes placed over the skin serves as the counterelectrode. A glucose sensing strip 16 is inserted into the foot ring 14, and the other end of the strip is inserted into a glucose meter 17. A cutter 12 is shown as a hollow tube that tapers near the tip to form a sharp cutting edge (such as a sharpened, even bottomed small hypodermic tubing stock).

FIG. 4 shows the sampling of interstitial fluid using an embodiment of the invention. Successive panels show the intact skin, appearance of interstitial fluid, fluid bolus contacting the cutter, and the lifting of the bolus by the tip of the cutter thus enlarging the bolus.

FIG. 5 shows the sampling of blood using an embodiment of the invention. Successive panels show the intact skin, appearance of blood, blood bolus contacting the cutter, and the lifting of the bolus by the tip of the cutter thus enlarging the bolus.

FIG. 6 shows measurement of blood glucose using interstitial fluid. The glucose strip is placed horizontally on the skin with the capillary opening located close to the cutter (left). As the interstitial fluid emerges from the skin, it is taken up by the capillary tube in the glucose strip (right).

FIG. 7 shows an alternate arrangement of the glucose strip, the strip is now placed vertically with the capillary in the strip contacting the skin (left). As the blood bolus appears from the skin, it is absorbed into the capillary tube (right).

### DETAILED DESCRIPTION OF THE INVENTION

The fluid collection method presented here results from the interaction of different forces acting on a volume of blood that appears from the opening made in the skin. The liquid contracts its surface area to maintain the lowest surface energy, causing it take a spherical shape. But, skin is not a flat, homogeneous surface; the uneven surface of the skin causes the blood drop to spread to a non-spherical shape. The surface tension of the blood drop determines its shape. A liquid drop (blood in this case) resting on a solid surface (skin) forms an angle called a contact angle between the liquid-solid interface and the liquid-vapor (air) interface. When a solid, such as a cutter, is immersed or pulled out of the blood drop, an advancing or receding contact angle is established. As the cutter is withdrawn from the blood, it imposes a force on the blood molecules, drawing them out along with the cutter. This, in turn, expands the volume of the blood bolus. The height to which the cutter deforms the blood drop depends on the wettability of the cutter (material make-up of the cutter), diameter of the cutter and the speed of retraction.

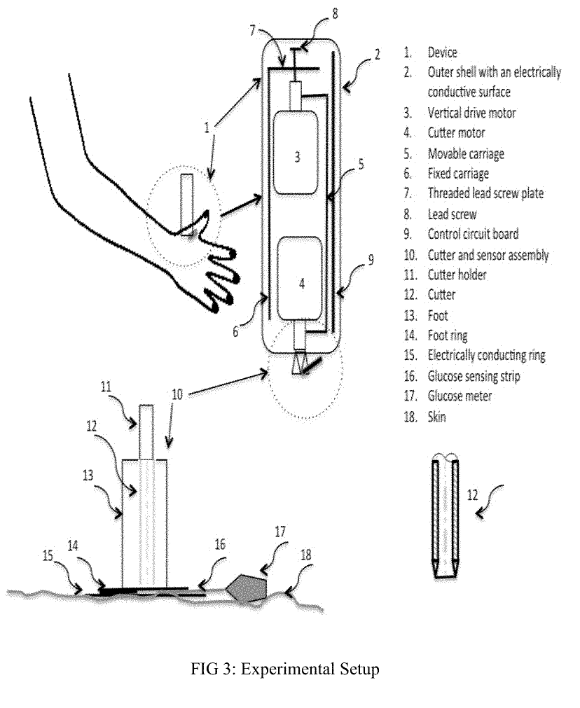

What follows here below are experiments performed using the apparatus and techniques described. These experiments are but a few of the possible applications and should not be viewed as a compressive list or discussion. The experiments were conducted with the setup shown in FIG. 3. The device 1 was placed on the back of hand (as an example site). The device 1 has two motors, 3 to drive the cutter 12 toward and away from the skin 18 and 4 to rotate the cutter around its long axis. A lead screw mechanism 8 is used to drive the movable carriage 6. The foot of the device 10 has a foot ring 14 that contacts the skin. An electrically conducting ring 15 is used as a counterelectrode. In certain embodiments a pair of ECG electrodes contacting skin 18 served as the counterelectrode. A glucose sensing strip 16 is inserted into the foot ring 14, and the other end of the strip is inserted into a conventional glucose meter 17.

### Examples

The following non-limiting examples serve to illustrate certain embodiments of the invention but are not to be construed as limiting. Variations and additional or alternative embodiments will be readily apparent to the skilled artisan on the basis of the disclosure provided herein.

### Example 1—Sampling of Interstitial Fluid

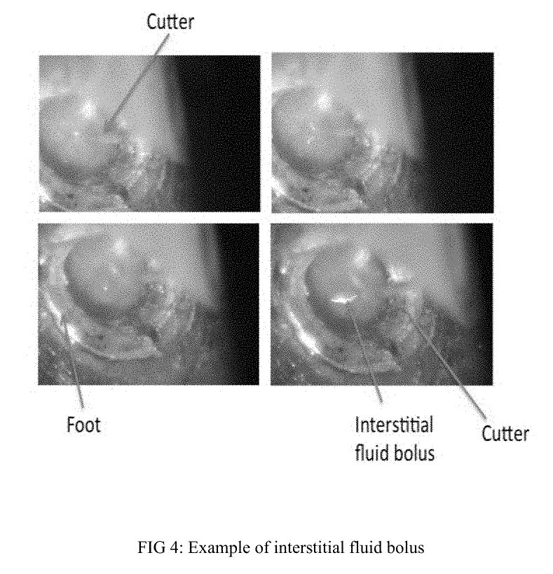

A 0.014″ diameter cutter with a short flute (0.015″ long) was mounted in a PathFormer device (1 of FIG. 3). The device uses skin impedance as a trigger to limit the depth of penetration into the skin. The device was used in the ‘dither’ mode in this experiment. A pair of Norotrode 20 electrodes stuck on the skin (away from the site of drilling) was used as counterelectrodes. The device was set to 15 kΩ trigger resistance. After the 10^{th} dither, clear interstitial fluid emerged from the skin. At the 30^{th} dither, there was a slight pricking sensation at which point a large bolus of interstitial fluid appeared (FIG. 4). Subsequently, the cutter started moving slowly into the bolus and drawing the fluid out. When the device was turned off and lifted off the skin, blood appeared from the site mixing with the bolus of interstitial fluid.

### Example 2—Sampling of Blood

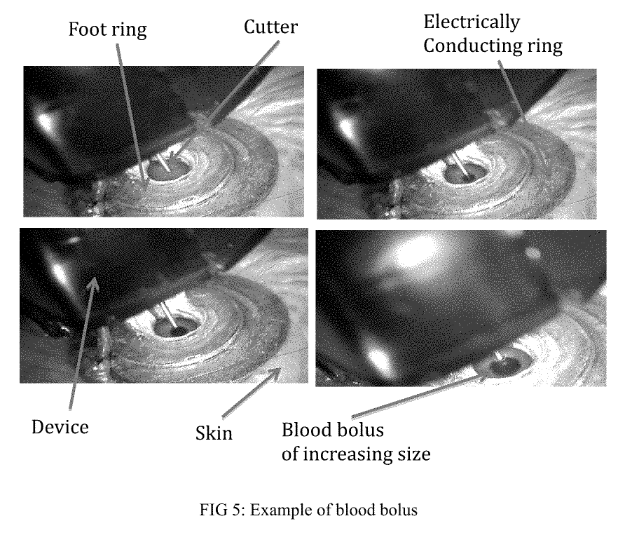

A 0.014″ diameter endmill was used as the cutter. The device was set to 15 kΩ trigger resistance. After 10 dithers, blood appeared via the opening, but the cutter did not appear to touch the skin during subsequent dithering (FIG. 5). The hole was clean and circular, but skin fragments were stuck to the cutter.

### Example 3—Sharpened Stainless Tube as Cutter

A stainless steel needle tube with its ends sharpened was used as the cutter (FIG. 5). The outside diameter of the tube was 0.024″. The tube was epoxied to a cylindrical brass holder with a 0.025″ hole through it. The back end of the tube was left open. The standard dither protocol was used: dither down into the skin until the preset trigger impedance (15 kΩ in this case) was reached, then retract and repeat the dither process. Within the first two dithers, blood started appearing, the cutter kept retracting, reaching down to the top of the blood bolus, pulling the bolus up, the contact between the bolus and the cutter breaks, the cutter goes down again. There was no sensation during the procedure. There was an ample bolus of blood at the end of the experiment (around 30 dithers). The cutter was clean on the inside, but there was a blood stain around the outside surface close to the end.

### Example 4—Abraded Cutter

A solid with a rough surface has a higher wettability than a smooth cutter. So, a 0.020″ diameter endmill was abraded by directing a stream of high speed aluminum oxide powder on it. The cutter cut into the skin on the first dither producing a bolus of blood. The bolus continued to increase in size over the next 10 dithers. The cutter subsequently reached the tip of the bolus and pulled back on every dither.

### Example 5—Integrated Blood Sampling and Blood Glucose Measurement—Vertically Aligned Strip

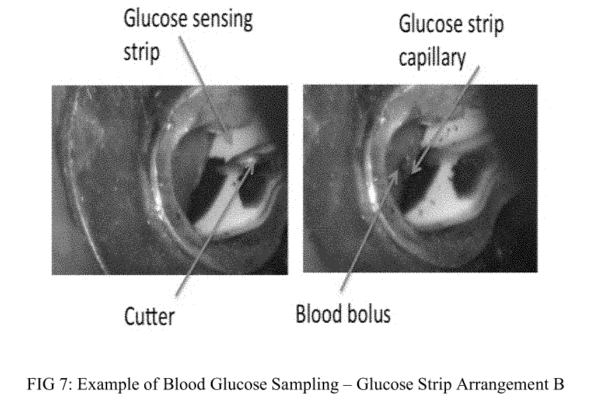

A 0.024″ diameter flat bottomed endmill was used as the cutter. A glucose strip was located close to the cutter by placing it vertically into a slot in the foot ring (FIG. 7). The glucose sensing strip was inserted into the glucose meter just before the drilling commenced. The device was operated in the dither mode with the retraction on low impedance being longer than the downward travel. Interstitial fluid started flowing out after the 4^{th} dither. The cutter started drawing the fluid out with every subsequent dither. The glucose strip was located away from the bolus, so the strip was moved closer to the bolus by the 23^{rd} dither, it took 10 more dithers to get enough fluid into the meter. The meter read 115 mg/dL at the end of the procedure. The cutter looked clean and sharp (although a bit more shiny) after drilling.

### Example 6—Integrated Blood Sampling and Blood Glucose Measurement—Horizontally Aligned Strip

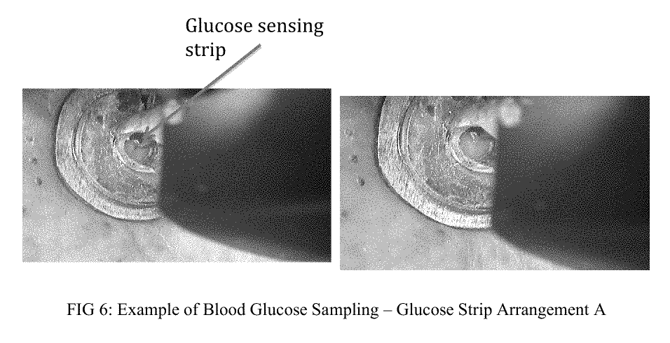

A stainless steel needle (23 gauge) with the end sharpened by abrading the outside and inside surface of the needle end was used (FIG. 6). The outside diameter of the needle was 0.024″. The needle was epoxied to a cylindrical brass holder with a 0.025″ hole through it. The back end of the needle was left open. The standard dither protocol was used: dither down into the skin until the preset trigger impedance (15 kΩ in this case) was reached, then retract and repeat the dither process. But, the drill stopped spinning after it encountered low impedance for the fifth time. A glucose strip was placed horizontally in the footer ring slot. The strip capillary tip was located very close to the needle cutter. There was very little blood from the dithering process. After around 1 minute, the strip had enough blood to make a measurement (91 mg/dL).

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

From the foregoing, it will be appreciated that although specific examples have been described herein for purposes of illustration, various modifications may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

What is claimed is:

1. A device for extraction of bodily fluids from tissue comprising a drilling device with a dithering cutter, wherein the cutter is an electrically conducting cutting element that breaches the stratum corneum and/or the epidermis of the skin, a drive assembly that moves the cutting element in a dithering manner, repeatedly into and away from the skin thereby forming a hole in the tissue, an analyte sensing element, and an electronic control module for controlling the skin cutting element and means for collection of the body fluids from the hole created in the tissue.

2. The device of claim 1, wherein the cutter rotates as it moves into and out of the tissue.

3. The device of claim 1, wherein the surface of the cutter is roughened to enhance the surface tension between the cutter and the body fluids, thereby improving the extraction of the bodily fluid from the tissue.

4. The device of claim 1, wherein the cutter comprises a hollow member that dithers in and out of the skin to create an opening and collect fluid in the bore of the cutter.

5. The device of claim 4, wherein the hollow cutter rotates as it moves into and out of the tissue.

6. The device of claim 1, wherein the cutter rotates prior to and during the process of creating a hole, but does not rotate subsequently.

7. The device of claim 1, wherein the cutter comprises a hollow cylindrical member into which the blood or interstitial fluid is drawn for subsequent analysis.

8. A method of extracting bodily fluids from tissue which comprises repeating the following steps using the device of claim 1:
moving the cutting element toward the skin in a dither mode wherein the cutter is moved in small steps toward and away from the skin with a small bias in movement toward the skin, monitoring an electrical impedance, and reversing the direction of movement when a preset level of decrease in the electrical impedance is detected; the procedure is repeated until a predetermined quantity of bodily fluid is collected.

9. The method of claim 8, wherein as the cutter moves into the tissue, it encounters fluid; as the cutter starts to retract, and move away from the skin, the cutter pulls the fluid up, thus enlarging the bolus of fluid at the surface of the hole.

10. The method of claim 9, wherein an analyte sensing element is positioned near the skin surface and proximate to the cutter, such that when the fluid is drawn out of the skin, it contacts the sensing element.

11. The method of claim 8, wherein the cutter rotates as it moves into and out of the skin.

12. The method of claim 11, wherein the rotating cutter creates an opening in the skin large enough to produce a drop of blood or interstitial fluid.

13. The method of claim 12, wherein the blood or interstitial fluid is drawn out of the hole by surface tension.

14. The method of claim 8, wherein a non-rotating cutter that dithers through the skin, creates an opening, subsequently draws blood or interstitial fluid out by surface tension.

15. The method of claim 8, wherein the surface of the cutter is roughened to enhance the surface tension between the cutter and the body fluids, thereby improving the extraction of the bodily fluid from the tissue.

16. The method of claim 8, wherein the cutter comprises a hollow member that dithers in and out of the skin to create an opening and collect fluid in the bore of the cutter.

17. The method of claim 16, wherein the hollow cutter rotates as it moves into and out of the tissue.

18. The method of claim 8, wherein the cutter moves in a dither manner until a sufficient amount of blood or interstitial fluid is collected for blood glucose measurement.
